(19) United States
(12) Patent Application Publication
(10) Pub. No.: US 2006/0057020 A1
(43) Pub. Date: Mar. 16, 2006

(54) Cleaning of air

(76) Inventor: Joseph Tufo, Epsom (GB)

Correspondence Address: NIXON & VANDERHYE, PC, 901 NORTH GLEBE ROAD, 11TH FLOOR, ARLINGTON, VA, 22203, US

(21) Appl. No.: 10/532,107

(22) PCT Filed: Oct. 21, 2003
(86) PCT No.: PCT/GB03/04540

§ 371 Date: Apr. 21, 2005

(30) Foreign Application Priority Data
Oct. 21, 2002 (GB) 0224427.5
Oct. 25, 2002 (GB) 0224897.9
Jan. 10, 2003 (GB) 0300563.4
Apr. 16, 2003 (GB) 0308808.5
Sep. 11, 2003 (GB) 0321324.6
Sep. 11, 2003 (GB) 0321325.3
Sep. 11, 2003 (GB) 0321326.1

(51) Int. Cl. A61L 9/20

(52) U.S. Cl. 422/24; 422/121; 96/224

(57) ABSTRACT

Apparatus (1) for cleaning air comprises a chamber (2) having an inlet (IL) and an outlet (OL), through which air to be cleaned is passable, a plurality of UV light sources (7) to irradiate the interior of the chamber (2) and one or more UV transmissible shield members (3, 4) isolating the UV light sources (7), in use, from the air to be cleaned and wherein at least some of the UV light sources (7) are mounted on a common mounting means such that said some of the UV light sources (7) are removable together from the apparatus (1). The apparatus (1) may comprise an upstream filter (9) and an air equalising grid (10). The grid (10) may have a UV reflective surface.

The present invention relates to apparatus for and a method of cleaning, for example purifying, air.

It has long been known that harmful agents can be borne by air. These harmful agents may include pathogens, such as micro-organisms, for example mould, bacteria and viruses, allergens, such as pollen, as well as dust and other detritus. Many solutions have been proposed to enable effective and efficient cleaning of air to remove such pollutants.

When air is re-circulated in buildings, there is a tendency for pollutants to become concentrated in the circulating air unless they are adequately removed. In buildings such as hospitals, schools and offices the presence of such pollutants can lead to acute and chronic illnesses and, in the worst cases fatalities. The same problem can arise in vehicles, e.g. aircraft, ships, other multiple passenger vehicles; in food processing areas; in mortuaries; and in the cultivation of certain crops such as mushrooms, beansprouts, asparagus, artichokes. The invention is applicable in all these situations.

In closed environments, such as aircraft, motor vehicles and like environments there is often a restraint on what can be installed because space is often at a premium. Other considerations such as the frequency of maintenance and replacing parts also have to be taken into account when installing air cleaning apparatus.

WO-A1-01/87362 (SUVAIR) discloses air disinfection apparatus having a chamber through which air can flow and an ultraviolet light source which irradiates the chamber to kill bacteria. The apparatus comprises an electrostatic filter adjacent the inlet to remove larger particulate matter. The UV light source may project into the chamber or may be located in a separate housing, the housing being separated from the chamber by a UV light transparent screen, which is fabricated from quartz or the like.

There are certain problems with the above-identified apparatus. Firstly, when the UV light source is located within the chamber, dirt and other detritus whose passage is not arrested by the filter, adheres to the source. Typically, such detritus is not UV transparent, so reducing the effectiveness of the light radiation. Secondly, it has also been found that the flow of air through the chamber cools the light source which concomitantly reduces the UV output of the lamp. To circumvent this problem it is necessary to increase the power supplied to the lamp which has a deleterious effect on lamp life. Thirdly, when the UV light source is located in a separate housing the amount of UV light which irradiates the chamber is reduced. In WO-A1-01/87362, UV reflective material lines the housing to reflect at least some of the impinging UV light back towards the chamber.

A further problem associated with WO-A1-01/87362 is associated with the use of an electrostatic filter. It has been found that such filters can arc during use. The electrical discharge is sufficiently energetic to dissociate oxygen and thereby lead to ozone production. As is known to the skilled addressee, ozone is an undesired pollutant in indoor atmospheres.

A further problem sometimes found is that the air duct may have to be shut down when replacing the UV light source(s).

DE-A1-4210509 (UERBERALL) discloses apparatus for disinfecting a fluid having a plurality of UV light sources mounted across a chamber through which a fluid may flow. Each light source is located within a quartz tube. The light sources are mounted in a spiral arrangement to maximise the irradiation of the fluid flow path. Where U-shaped light sources are used, they are configured so that the radiation maxima are not parallel or normal to the fluid flow direction.

Such an arrangement has been found to present problems when replacing lamps and/or servicing the interior of the apparatus.

JP 11197229 (HONDA) discloses an air cleaner comprising a fan, a photocatalyst filter and a plurality of UV lamps. Each lamp is mounted in a ‘wind shield’ which has an ‘upstream’ UV transparent side to prevent temperature reduction associated with the contact of the lamps with the air.

Problems may be associated with the cleaner of HONDA because of the proximity of the fan to the filter (which may cause uneven flow through the cleaner) and the lack of downstream filtration. The cleaner of this reference is not suitable in many locations.

Accordingly, it is an object of this invention to reduce and ideally eliminate one or more of the problems found with prior art air cleaners. It is a particular object to provide apparatus which can be fit in a duct, or retro-fit to an existing duct or used in a portable system. A further object of the invention is to provide a cleaner for air which has a substantially equalised volume of air to be cleaned entering the apparatus. Other advantages of this invention will become apparent from the following.

A first aspect of the invention provides apparatus for cleaning air, the apparatus comprising a chamber, having an inlet and an outlet, through which air to be cleaned is passable, a plurality of UV light sources to irradiate the interior of the chamber and one or more UV transmissible shield members isolating the UV light sources, in use, from the air to be cleaned and characterised in that at least some of the UV light sources are mounted on a common mounting means such that said some of the UV light sources are removable together from the apparatus.

There is further provided, in a second aspect of the invention, apparatus for cleaning air, the apparatus comprising a chamber having an inlet and an outlet, through which air to be cleaned is passable, a plurality of UV light sources to irradiate the interior of the chamber and one or more UV transmissible shield members isolating the UV light sources, in use, from the air to be cleaned and filter means upstream of said UV light sources, and characterised in that upstream of said filter means is located air flow equalising means which, in use, ensures that the flow of air through said filter means is substantially constant across the area of said filter means.

Preferably, at least the downstream surface of said air flow equalising means is at least partially UV reflective.

Said air flow equalising means may comprise a downstream surface which comprises a plurality of angled air flow paths, the angle of which preferably being 45° to the major flow axis of air passing through the apparatus.

Preferably, said air flow equalising means comprises a plural—(e.g. three) layered structure, each layer comprising a plurality of angled flow paths. The flow paths of adjacent layers preferably are differently angled to encourage, in use, air flowing therethrough to adopt a tortuous flow path.

A yet further aspect of the invention provides apparatus for cleaning air, the apparatus comprising a chamber having an inlet and an outlet, through which air to be cleaned is passable, a plurality of UV light sources to irradiate the interior of the chamber and one or more UV transmissible shield members isolating the UV light sources, in use, from the air to be cleaned and filter means upstream of said UV light sources, and characterised in that upstream of said filter means is located a UV reflector arranged to allow air to flow therethrough but to prevent UV light from passing therethrough at least a portion of incidental UV light being reflected towards said filter means.

Preferably, the UV transmissible shield member or members is/are formed from quartz, fused silica, UV transmissible plastics or other suitable materials.

Preferably, the UV light sources are UV lamps, emitting light in the UV-C band (typically 254 nm). Suitable UV lamps are elongate tubes. It has been found that for good effect the lamps should have a non-circular cross-sectional shape, say oval with flats. This increases the effective area of radiation. Preferably the lamps are arranged in banks, say each of four lamps. Preferably the mean level of radiation is above 10 milliwatts mean level (ie 10 mW cm⁻²). In a preferred embodiment the mean level of radiation is 10-70 mW cm⁻², say 60-70 mW cm⁻², although the radiation level may be higher than 70 mW cm⁻². The mean level of radiation is the average of the measured radiation level in a line along the centre of the chamber aligned with the principal flow path axis of gas to be cleaned.

In one embodiment the shield member provides at least part of a boundary defining the flow path of the air to be cleaned.

The shield member may be a tube mounted within the chamber, the air to be cleaned being flowable thorough the tubular shield member, the UV light sources being located between the chamber wall and the shield member.

The shield member may provide at least a part of at least one portion of the chamber, say one wall thereof, the UV light sources being located outside of the to-be-cleaned air flow path.

In a second embodiment, the shield members may comprise tubes which extend across the chamber, a UV light source being mounted within each of the tubular shield members.

The chamber may comprise one or more walls which comprise a UV reflective surface, such as quartz mirrors, or polished aluminium or the like. One or more walls comprising a UV reflective surface may be shaped to present a concave surface (as viewed from the major axis of the flowing air), say to form a parabolic or other reflector.

Where the UV light sources are located outside of the chamber, they may be mounted adjacent a UV reflective surface to reflect impinging UV radiation into the chamber.

The apparatus may also include first filter means situated across the inlet and, preferably, second filter means situated across the outlet. Said first filter means may comprise a relatively coarse filter element and said second filter means may comprise a HEPA filter element. Extra filter elements may be present at the inlet and/or the outlet. For example, an activated carbon filter may be present downstream of the chamber, say downstream of a HEPA filter.

Preferably one or both of the relatively coarse and HEPA filter elements are fabricated from a UV transmitting material. Said second filter means may comprise a frame, preferably a combustible frame, such as one fabricated from wood, a derivative of wood or the like.

The relatively coarse filter may be coated and/or impregnated with an antimicrobial or biostatic substance.

A component made of a material which releases hydroxyl radicals known to be detrimental to pathogens may be present. Such a material may be a metal oxide such as titanium dioxide, which may be coated on a screen upstream of the UV sources and/or on the relatively coarse upstream filter.

In a preferred embodiment, the apparatus comprises two banks of four UV lamps each bank being located at opposed sides of the flow path of the air to be cleaned, the banks being separated from the flow path by a UV transmissible shield member in the form of a plate of quartz, each bank being removable as a unitary structure.

Preferably, the apparatus comprises one power supply for two or more lamps.

Preferably, the or each power supply is tunable to deliver less or more power to the lamps to which it is connected. The apparatus may comprise UV monitoring means arranged to control the or each power supply to increase or decrease the power supplied, thereby ensuring that the UV irradiation within the chamber is monitored and/or kept within tolerable limits.

A fourth aspect of the invention provides apparatus for cleaning air, the apparatus comprising a chamber, having an inlet and an outlet, through which air to be cleaned is passable, a plurality of UV light sources to irradiate the interior of the chamber and one or more UV transmissible shield members isolating the UV light sources, in use, from the air to be cleaned and characterised in that two or more lamps are supplied with power from a tunable power source, said two or more lamps having UV monitoring means associated therewith, said UV monitoring means being arranged in operative feedback control of the power source to ensure UV output from the lamps is within tolerable limits.

A fifth aspect of the invention provides a portable air cleaning unit, the unit comprising a housing in which is located air cleaning apparatus as hereinbefore described and air movement means operable to cause air to flow from the inlet to the outlet of the air cleaning apparatus.

A yet further aspect of the Invention provides a method of cleaning air, the method comprising moving air through any of the air cleaning apparatus as hereinbefore described and expelling cleaned air.

The method may further comprising reflecting UV light from a upstream surface of an air flow regulating means onto a downstream surface of filter means located upstream of the UV light source.

The invention may be applied to a variety of situations, as will be described below. One situation where it is necessary to isolate an individual from a package which may be suspect in as much as it may be thought to contain a hazardous substance.

A further aspect of the invention provides the invention provides a method of treating a package to release hazardous material which is associated with the package, the method comprising introducing pressurised air which is free of dust particles and micro-organisms into a zone of a chamber containing the package to be treated, contacting the air with the package, and releasing hazardous material therefrom.

Preferably the air which has passed over the package is then passed over an ultraviolet light source to treat particles of hazardous material released into the pressurised air.

The method most preferably includes the step of locating in a lower region of the zone an ultraviolet source and allowing or causing particles of the released hazardous material to fall thereon.

Preferably a negative pressure is maintained within the chamber.

The pressurised air is passed through the chamber at a suitable rate.

Preferably, a table is present in the zone to receive the package, the table being perforated so that the pressurised air can pass therethrough.

Preferably gloves are present in the zone and accessible from the outside by which an operator may manipulate a package on the perforated table to cause or allow release of hazardous material.

In another aspect the invention provides apparatus for treating a package to release and destroy hazardous material which is associated with the package, the apparatus comprising a chamber having a zone containing the package to be treated, means for supplying air which is pressurised and free of dust particles and micro organisms to the zone to contact that air with a package in the zone, and an ultraviolet light source in a lower region of the zone to destroy such hazardous material.

Preferably the apparatus includes an ultraviolet light source to destroy particles of hazardous material retained in the pressurised air after it has passed over the package.

Preferably the apparatus includes means to maintain a negative pressure in the chamber.

A yet further aspect of the invention provides a room having located therein apparatus as hereinbefore described and means operable to move air from the inlet to the outlet of the apparatus and further comprising valve means operable to provide a negative or positive pressure in the room.

There is further provided, in a further aspect of the invention, air cleaning apparatus, the apparatus comprising a chamber, having an inlet and an outlet, through which air to be cleaned is passable, a plurality of UV light sources to irradiate the interior of the chamber and one or more UV transmissible shield members isolating the UV light sources, in use, from the air to be cleaned and characterised in that the UV light sources have an oval cross-section and are arranged to provide, in use, a mean level of radiation in excess of 10 mW cm², preferably in excess of 60 mW cm⁻².

The invention will now be described, by way of example only, and with reference to the accompanying Figures in which:

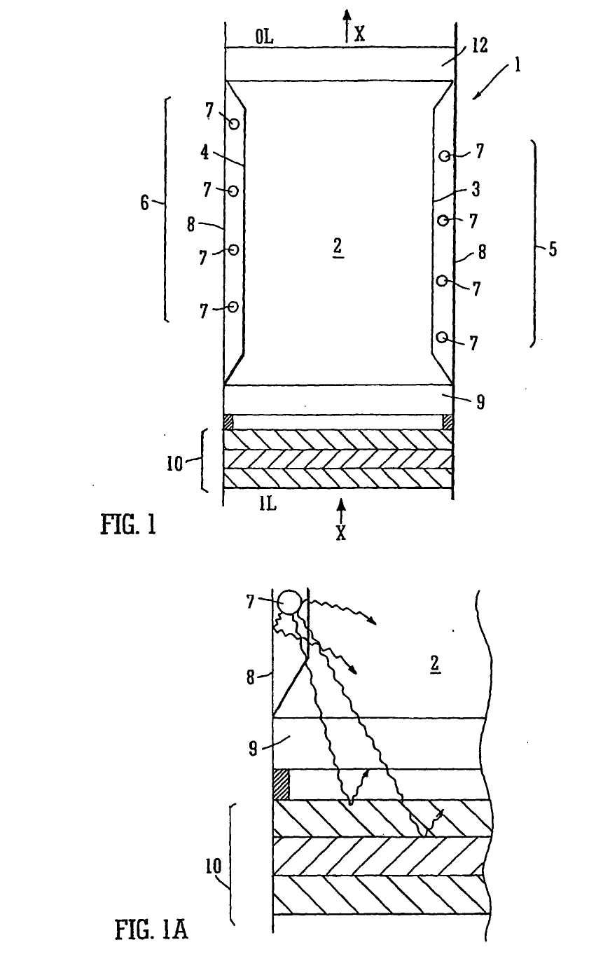

FIG. 1 is a sectional elevation of a first embodiment of air cleaning apparatus according to the invention;

FIG. 1A is a sectional elevation of a part of the apparatus of FIG. 1;

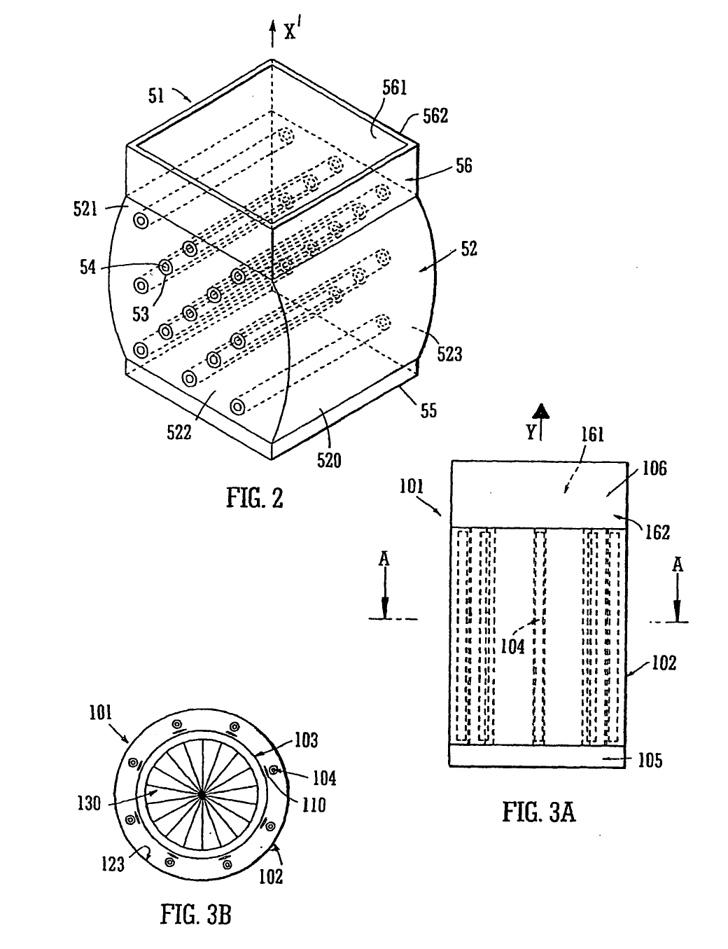

FIG. 2 is a perspective view of a second embodiment of air cleaning apparatus according to the invention;

FIG. 3A is an elevation of a third embodiment of air cleaning apparatus according to the invention;

FIG. 3B is a view along line A-A′ of FIG. 3A;

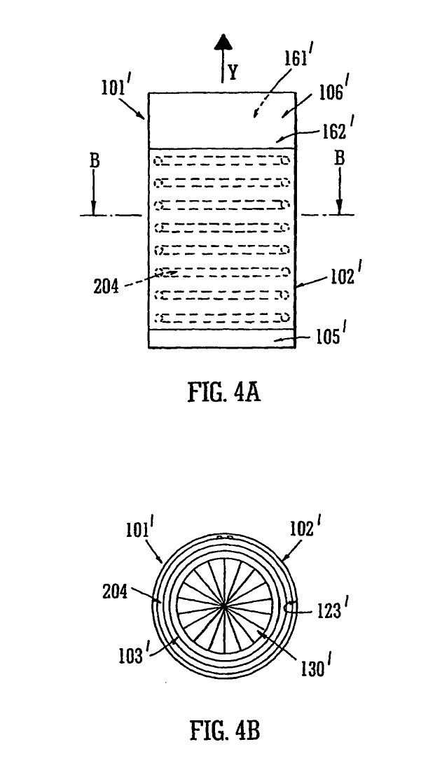

FIG. 4A is an elevation of a variation of a fourth embodiment of air cleaning apparatus according to the invention;

FIG. 4B is a view along line B-B′ of FIG. 4A;

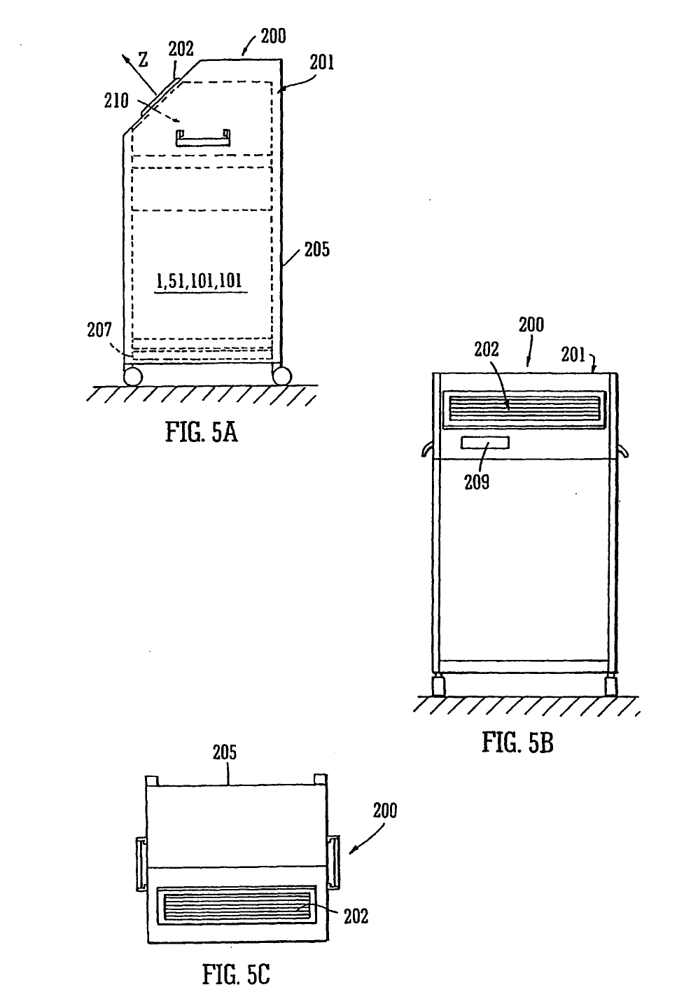

FIG. 5A is a side elevation of a unit according to the invention;

FIG. 5B is a front elevation of the unit of FIG. 5A; and

FIG. 5C is a plan view of the unit of FIG. 5A.

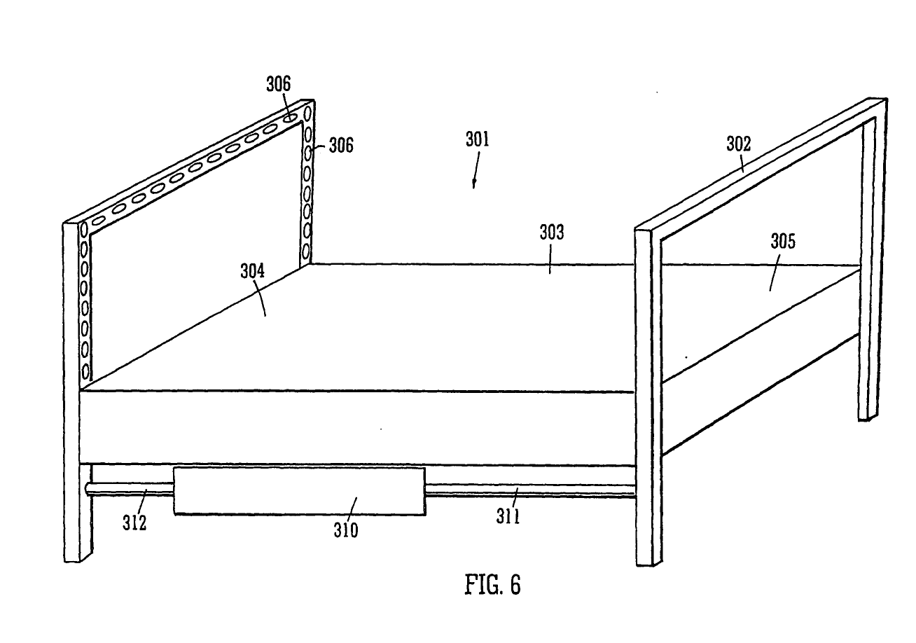

FIG. 6 is a perspective view of a bed incorporating apparatus according to the invention;

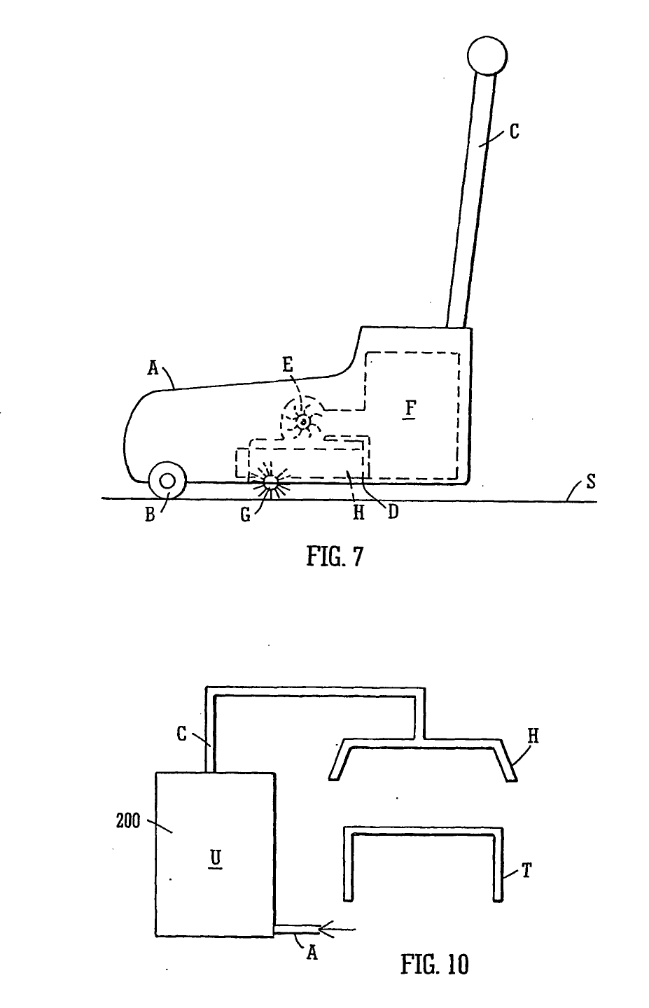

FIG. 7 is a schematic illustration of vacuum cleaning apparatus including apparatus according to the invention;

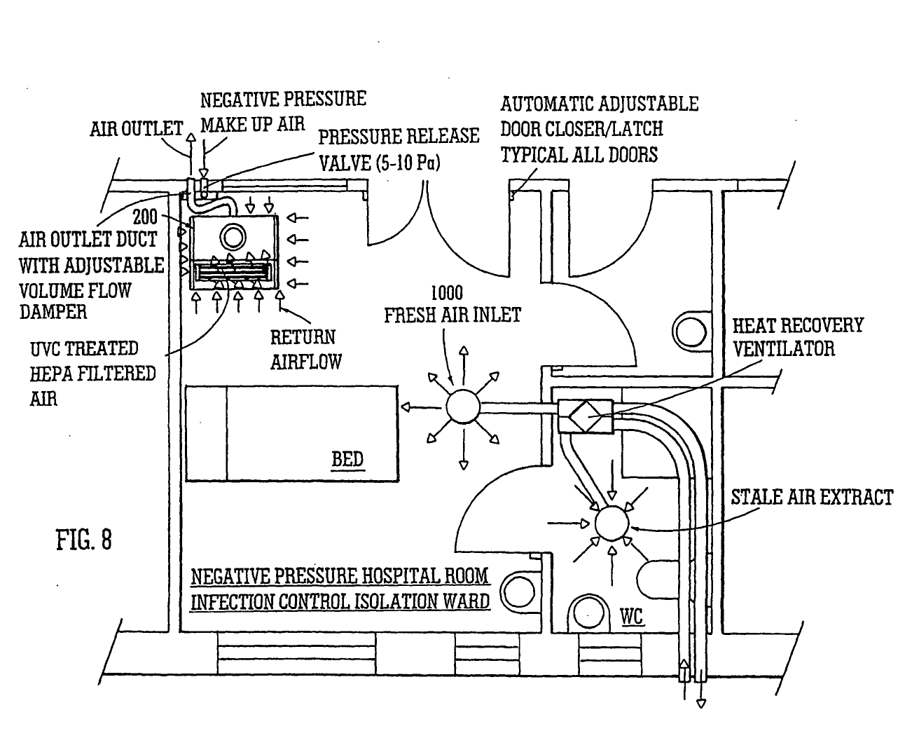

FIG. 8 is the layout of a hospital room having a negative pressure room;

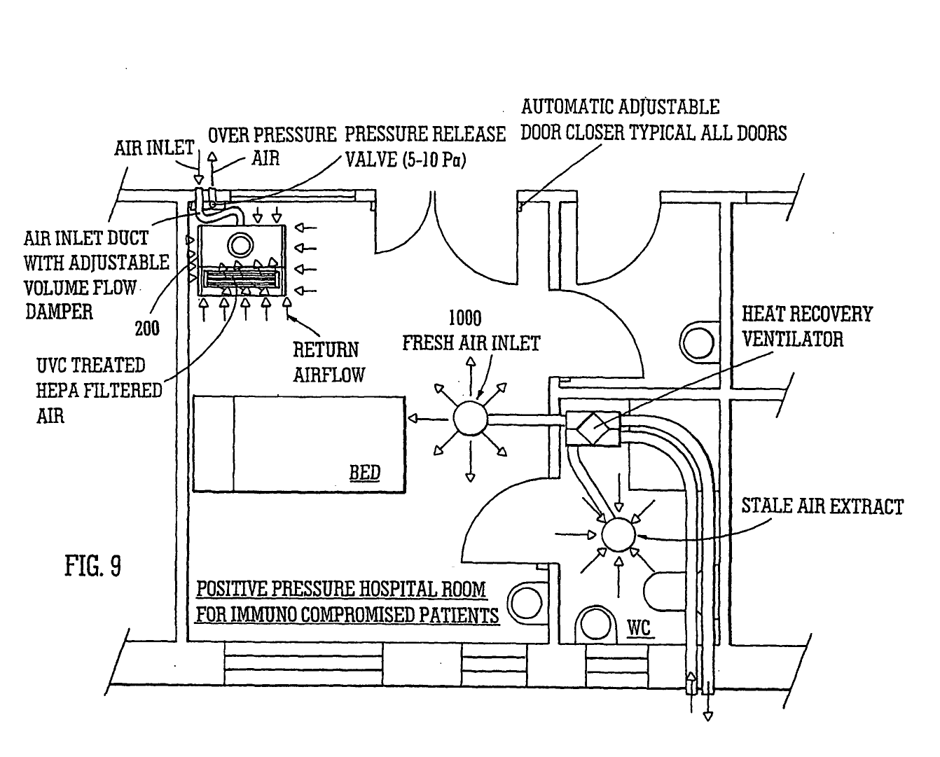

FIG. 9 is the layout of a hospital room having a positive pressure room;

FIG. 10 is a schematic illustration of an operating theatre;

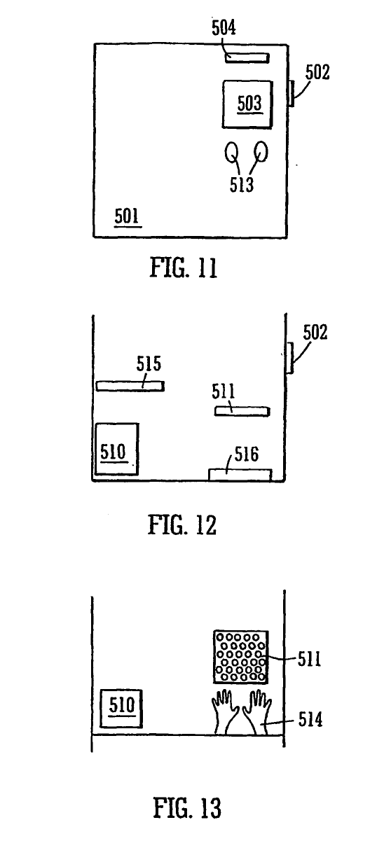

FIG. 11 is an elevation of a cabinet incorporating the unit of the invention;

FIG. 12 is a vertical section through the cabinet of FIG. 11, and

FIG. 13 is a transverse section taken on lines III-III shown on FIG. 12.

Referring to FIGS. 1 and 1A, there is shown air cleaning apparatus 1, having a rectangular (e.g. square) chamber 2 through which air to be cleaned is flowable in the direction of arrows X from an upstream inlet IL in to a downstream outlet OL.

At least part of two opposed walls 3, 4 of the chamber 2 are formed of UV transmissible material, for example quartz. A bank 5, 6 of four UV-C lamps 7 is mounted adjacent each of the opposed walls 3, 4. The lamps 7 are located between the wall 3, 4 and a UV reflective surface 8.

Upstream of the lamps 7 thee is mounted a coarse pre-filter 9 which is at least partially UV transmissible. The filter 9 may be coated or impregnated with an antimicrobial substance such as Biogreen 2000 (supplied by KCM Limited of Croydon, United Kingdom). Because the filter 9 is at least partially UV transmissible, UV radiation may penetrate the filter 9 to irradiate any substances trapped therein.

Located upstream of the filter 9 is a flow equalising grid 10 which is fabricated from three layers of angled louvers formed of UV reflective material. The louvers of adjacent layers of the grid 10 are alternately angled which forces air passing therethrough to adopt a tortuous path and has the effect of equalising the speed of the flow of air. Also, because at least the upper layer of the grid 10 is formed of UV reflective material, any UV impinging on the grid 10 is reflected onto the downstream surface (the lower surface as shown) of the filter 9.

The grid 10 is separated from the filter 9 by a spacer element 11.

Because the grid 10 is formed of three layers of alternately angled louvers (each preferably being angled at 45° to the principal flow axis of air), no UV light can be emitted from the grid. The grid 10 could be fabricated from two or more layers. However, three has been found to be suitable because, inter alia, it has been shown to provide adequate protection and economy of space.

Located downstream of the light sources 7 is a HEPA filter 12.

Both filters 9, 12 are standard 600×600 mm filters.

Each bank of lights 5, 6 is associated with a pair of power supplies (not shown), one for the lower pair of lamps 7 in a bank (5 or 6) and one for the upper pair of lamps 7.

The lamps 7 of a bank of lamps 5, 6 may all be mounted on a common mounting (not shown) or the pairs of lamps of each bank 5, 6 may be mounted on a common mounting (also not shown).

Thus, to replace lamps 7 one simply needs to gain access to the supply, unplug the power supplies from the lamps 7 and slide out the common mounting with the (2 or 4) lamps 7 attached thereto.

In use, air to be cleaned is drawn or formed through the apparatus 1 in the direction of the arrows X from the inlet IL to the outlet OL. The air passes in tortuous fashion through the grid 10 and is filtered by the filter 9 which removes larger particles.

The air is irradiated by UV radiation emitted from the lamps 7, either directly through the quartz walls 3, 4 or reflected from the reflective surfaces 8 and then through the quartz walls 3, 4.

The irradiated air then flows through a UV transmissible HEPA filter 12 to the outlet OL.

An optional activated carbon filter (not shown) may be located downstream of the HEPA filter 12.

When the air flow through the apparatus 1 is stopped, the lamps 7 continue to emit for a short period (e.g. 1-3 minutes) to ensure that anything trapped on the filters 9, 12 is fully irradiated.

Using oval lamps 7 of principal cross-sectional dimension 4 to 5 mm and 20 to 30 mm (e.g. 4.5 mm and 25 mm) a mean UV intensity of greater than 60 μm ² (˜60 mW cm⁻²) was achieved.

The lamp 7 operated at a temperature of about 100-120° C.

In comparison, identical lamps 7 operated without UV transmissible walls 3, 4 (i.e. the lamps 7 were exposed to the cooling effect of the air flow) were only able to operate at about 80° C., had a much lower UV output (approx. 80%) and required about 30% greater energy input.

To test the efficiency of the apparatus 1, a particle test according to BS3928 0.3 μm was conducted in which NaCl particles of 0.3 μm particle size were released into a 100 m³ test chamber. The following results were obtained:

| Fan Speed | Air Vol/m³ hr⁻¹ | Removal % |
|---|---|---|
| Ultra Low | 600 | 99.99 |
| Low | 930 | 99.99 |
| Medium | 1260 | 99.99 |
| High | 1600 | 99.99 |

Test were also conducted to determine the efficiency of bacteria removal. Twenty three Bucillus subtilis hazards were released into a 100 m³ test chamber over the course of five days. The results at 10⁷ spores were as follows:

| | | | Removal |
|---|---|---|---|
| | Time to half life | Time to 90% | efficiency |
| Air Volume/m³ hr⁻¹ | reduction/min | reduction/min | after 30 min/% |
| 600 | 7.5 | 23.0 | 96.50 |
| 930 | 5.0 | 15.9 | 98.30 |
| 1260 | 4.5 | 14.5 | 98.35 |
| 1600 | 4.0 | 12.7 | 97.49 |

In order to ensure proper operating of the apparatus 1, a plurality of sensors (not shown) are included. One such sensor monitors the UV output of each of the lamps 7. If a particular lamp's output drops outside of a tolerable limit, a signal is sent to an on-board computer (OBC, not shown) which increases the power supply to the lamp 7, thereby increasing the output of the lamp 7.

Other sensors include pressure sensors across the filters 9, 12 and air flow sensors. If any of the sensors detect a signal outside tolerable limits an alarm may be sounded. The alarm signal may be sent to a remote site via a radio link or via an on-board modem.

Referring to FIG. 2, there is shown air cleaning apparatus 51 having a chamber 52 in which is located a series of quartz sleeves 53 (13 being shown, but the number may be raised, and range from say 8 in two banks each of 4 lamps). A UV-C lamp 54 is located within each sleeve 53. At one end of the chamber 22 there is mounted filter 55 across the inlet to the chamber 52. Below the filter 55 is a UV screen 59 similar to the grid 10 described above. At the other end of the chamber 52, across the outlet thereof, there is mounted a HEPA filter 56.

The lamps are arranged to emit light in the UV-C band of about 254 nm. The output is above 10 mWcm⁻² mean level.

Two opposed walls 520, 521 of the chamber 52 have a concave inner surface, the other two walls 522, 523 being planar. Walls 520, 521 are formed from, have applied thereto, or are coated with aluminium, which is polished or shiny to enhance the UV reflectivity of the walls 520, 521. The planar side walls 522, 523 are similarly treated.

In use, air is drawn or pushed through the apparatus in the direction of arrow X′ by fan means (not shown). Thus, air is pre-filtered by the filter 55 to remove relatively large particulate material and enters the chamber 52 where it is irradiated by the UV-C lamps 54. It will be appreciated that the UV-C radiation is emitted through 360° and, because of the number and arrangement of the lamps, maximum irradiation of the incoming air is ensured. The UV-C light is also reflected from the walls 520, 521, 522, 523 to ensure complete irradiation of the space defined by the walls 520, 521, 522, 523. The concave surfaces of walls 520, 521 act as parabolic reflectors to the UV-C radiation. The radiation cannot escape from the chamber, so reducing the risk of injury to an operator. As the air is drawn or pushed through the chamber 52 the transported viruses, mould and bacteria are killed or rendered inert by the actinic radiation.

The filter 55 may be formed of a UV transmissible material. It may also have one or both of its major surfaces coated with an anti-microbial or biostatic substance.

After passing through the UV irradiation zone, the air is finely filtered by the HEPA filter 56. Any organisms which have not been affected by the UV-C radiation, are trapped on or in the HEPA filter 56. The HEPA filter element 561 is preferably made from a UV transmissible material such as glass fibres. Therefore, any trapped bacteria, mould or virus undergoes further irradiation, ensuring that it is rendered non-viable. The HEPA filter element 561 may be held in place by a frame 562. Preferably, the frame 562 is fabricated from a combustible material, such as wood. Once the nominal lifetime of the filter 56 has elapsed, the whole filter 5 may be removed and incinerated. The frame 562 makes it easy for an operator to remove and replace the filter 6. Another filter may be present, e.g. of carbon particles.

During operation of lamps 54, there is a tendency for a static and/or magnetic field to build up, which usually attracts dust and dirt particles. These fall on the sleeves instead of on the lamps 54. The sleeves 53 also ensure that a degree of turbulence is induced into the air flowing through the chamber 52.

It will be appreciated that to change a lamp 54 during routine maintenance it is unnecessary to turn off the power to all of the lamps 54. The emissive part of each lamp 54 is within the chamber 52, thus an operator may simply disconnect the power supply to the lamp 54 which is to be changed and slide that lamp 54 out of its respective sleeve 53. The procedure is reversed to install a new lamp. This is advantageous because there is reduced down-time for the apparatus and during lamp replacement there is a reduced risk of exposure of the operator to UV-C radiation.

It is most preferred that the lamps 54 are mounted on common mounting means (not shown) in two or three banks so that a plurality of lamps 54 are removable together.

During routine maintenance of the apparatus 51, the sleeves 53 may require cleaning. To do so it is necessary to cut off power to the unit 15. The sleeves 53 can then simply be removed and wiped clean.

It is a feature of the invention that when the apparatus is switched off the lamps are energised for about 1-5 minutes and continue to operate after the fan is switched off. This reduces the risk of injury to the operator. Dual safety circuits and UV-C protected inspection windows are present to confirm that lamps are switched off.

FIGS. 3A and 3B show a third embodiment of air cleaning apparatus 101 having a chamber 102 mounted within which elongate UV-C lamps 104 (8 being shown), are circumferentially spaced about the inner periphery of the chamber 102, with their axes parallel to that of the chamber 102. A quartz sleeve 103 is mounted co-axially within the chamber 102, the lights being located between the sleeve 103 and the wall of the chamber 102. If it is desired that a greater intensity of UV light is to be directed to the centre of the chamber 102, a quartz focussing lens 110 may be located adjacent each lamp 104.

A filter 105 is mounted across the inlet to the chamber 102. At the other end of the chamber 102, a HEPA 106 extends across the outlet.

The internal surface 123 of the wall of the chamber 102 is formed from, is coated with, or has placed thereupon aluminium, which is polished.

Mounted upstream of the filter 105 is a fixed blade fan 130. The fan 130 induces turbulence in any air which flows thereby. In some embodiments, the fan 130 will be replaced with a grid 10 as previously described.

In use, air is drawn or pushed through the apparatus 101 in the direction of arrow Y by fan means (not shown). Thus, air passes by the fan 130 which induces turbulence in the air flow. The air is pre-filtered by the filter 105 to remove relatively large particulate material which air then enters the chamber 102 where it is irradiated by the UV-C lamps 104. The UV-C light is also reflected from the walls 123 to ensure complete irradiation of the space defined by the sleeve 103. As the air is drawn or pushed through the chamber 102 viruses, mould and bacteria are killed or rendered inert by the actinic radiation.

The filter 105 is preferably formed of a UV transmissible material such as glass fibres. It may also have one or both of its major surfaces coated with an anti-microbial biostatic coating.

After passing through the UV irradiation zone, the air is finely filtered by the HEPA filter 106. Any viable mould, bacteria or viruses which have not been killed by the UV-C radiation, will be trapped on or in the HEPA filter 106. The HEPA filter element 161 is preferably made from a UV transmissible material such as glass fibres. Therefore, any trapped bacteria, mould or virus undergoes further irradiation, ensuring that it is rendered non-viable. The HEPA filter element 161 may be held in place by a frame 162. Preferably, the frame 162 is fabricated from a combustible material, such as wood. Once the nominal lifetime of the filter 106 has elapsed, the whole filter 106 may be removed and incinerated. The frame 162 makes it easy for an operator to remove and replace the filter 106, and aids in the incineration process.

During operation of lamps 104, there is a tendency for a static field to build up, which usually attracts dust and dirt particles. The sleeve 103 prevents the build up of dust or other detritus on the lamps 104.

The lamps 104 are preferably mounted on one or more common mountings (not shown) so that two or more lamps 104 may be removable together. The power to the lamps 104 will be supplied as described above.

FIGS. 4A and 4B show a variation of the apparatus of FIGS. 3A and 3B, wherein identical components are indicated by the same numerals with the addition of a prime (′).

The apparatus 101′ has all of the components previously described with the exception of the lamps 104. In the apparatus 101′, a series of toroidal or omega shaped lamps 204 (8 shown) are located along the major axis of the chamber 102′ in the space defined between the quartz sleeve 103′ and the inner wall of the chamber 102′. As will be appreciated, operation of apparatus 101′ accords with that of 101.

Four of lamps 204 may be located in a single mounting (not shown) for slidably removing the lamps 204 during maintenance.

The apparatus 1, 51, 101, 101′ may be used to clean air in a fixed system, such as those used in buildings, vehicles and the like, wherein air from, say, a room is exhausted via ducting which is connected to the inlet of the apparatus 1, 101, 101′. The so-cleaned air is then returned to the, say, room either directly or via a further air conditioning plant, for example a heat exchanger to warm or cool the air.

Several air-cleaning apparatus 1, 51, 101, 101′ may be connected in parallel so that cleaning or routine maintenance of one may be carried out whilst the others are operating.

The apparatus 1, 51, 101, 101′ may also be used in a portable air-cleaning unit 200, as shown in FIGS. 5A, 5B and 5C. The unit 200 has a housing 201 in which is located the air cleaning apparatus 1, 51, 101, 101′ according to the invention. Fan means 210 is located within the housing 201 adjacent the outlet of the apparatus 1, 51, 101, 101′ to draw dirty or un-purified air through the apparatus 1, 51, 101, 101° and expel cleaned air in the direction of arrow Z via a grille or screen 202.

A power supply will be provided to power the various components of the unit 200. A logic circuit device may be present to detect dirty filters, component failure, power interruption, and the like and to cause a fail safe shut down.

In a preferred embodiment, all of the onboard monitoring devices are monitored by an OBC or other logic circuit device. The OBC communicates with a remote site using GSM wireless technology. Failure of any component, or an unusual reading (e.g. the presence of a pollutant/contaminant), will be notified to the remote monitoring site via the wireless link. Also, routine monitoring of the apparatus can be carried out without the need to send a technician to the site. Thus the following (non-exclusive) list of parameters may be monitored and subsequently transmitted to the remote site: the UV intensity in the chamber, condition of the lamps, lamp countdown lifetime, air speed through the chamber, pressure drop across the or each filter, filter condition, total volume of air cleaned. The apparatus may also contain solid state monitors for pollutants such as anthrax or other undesired contaminants which may be monitored to identify the presence of any such species. Other monitors may also be present.

The unit 200 may have a baffle 207 mounted within the housing 201 adjacent the inlet of the apparatus 1, 51, 101, 101′ to prevent any UV-C radiation form exiting the housing 201.

Preferably, the unit 200 will be provided with sensors to monitor the pressure drop across the filters of apparatus 1, 51, 101, 101′. As the pressure drop increases it is evidence that a filter element is becoming occluded with matter. The signals from the sensors can be compared with pre-calibrated readings to enable an OBC or other comparitor to activate a warning signal when the filter or filters require replacement. The unit may also have sensors to monitor the output of the sensors monitoring the lamps to activate a warning signal upon failure of the lamps. A lamp-lifetime countdown timer may also be provided to monitor the time of use of the lamps. An access panel 205, which affords access to the apparatus 1, 101, 101′ may be interlocked to the energy source for the lamps. Thus, opening the panel will cause the energy supply to the lamps to be interrupted.

The unit 200 may be provided with fan means to push air through the apparatus 1, 51, 101, 101′. Any suitable device may be used, such as a fan or other device known to be useful in moving air.

The apparatus of the invention 1, 51, 101, 101′ is space-efficient, effective in destroying potential pathogens and in cleaning air. The presence of a plurality of light sources, each being shielded from the direct flow of the air confers many advantages. The apparatus may be retrofit into existing air-cleaning apparatus.

The invention is not limited to the embodiments shown. Baffles may be present to prevent UV radiation leakage such as a UV screen at the inlet and the outlet. A carbon filter may also be present at the outlet. Electronic controllers may be present to monitor the temperature of the lamps and intensity.

Specific, but not exclusive uses to which the air cleaning apparatus 1, 51, 101, 101′ may be put to use will be described below.

Referring to FIG. 6, there is shown a bed 301 having a frame 302 and a mattress 303. The frame has an intended ‘head’ end 304 and an intended ‘feet’ end 305.

The frame 302 is perforated with a plurality of apertures 306 at both the head and feet ends 304, 305.

Located beneath the bed 301 is air cleaning apparatus 310, having an upstream end connected to the feet end 305 of the frame 302 by tubing 311 and a downstream end connected to the head end 304 of the frame 302 by tubing 312.

The air cleaning apparatus 310 will typically accord with that shown in FIGS. 3A and 3B with the downstream end of the apparatus 101 being adjacent tubing 312.

In use, air is drawn through the apparatus 101 where it is filtered and cleaned of pollutants by filters 105, 106 and the UV lights 104. The cleaned air is passed along tubing 312 and into the hollow frame 302 at the head end 304 of the bed 301. The dean air is emitted from the apertures 306 to create a clean-air shroud over at least the head end 304 of the bed such that, any patient lying in the bead will breathe air from the clean air shroud emitted from the frame and maintained at the head end of the bed 301.

Air may be drawn into the apparatus via apertures 306 in the frame at the foot end 305 of the bed 301.

In a further embodiment, air may be drawn from any location into the apparatus 310.

In a preferred embodiment, the apparatus 101 only comprises one or two UV lights 104.

The drawings shows what may be described as a bespoke bed 301 wherein the frame 302 is used to emit cleaned air. However, it is also possible to clip apparatus 310 according to the invention to any bed and use one or more outlet pipes to ensure that the cleaned air is directed into the breathe-space of the individual.

In a further embodiment, the apparatus 310 may be used to draw air from the ‘breathe-space’ of an individual, clean the air and emit cleaned air into the ‘breathe-space’ of the individual to ensure that the individual is breathing in cleaned air and that the exhaled air is cleaned.

The apparatus may be used in a bed or in a chair, for example where clean air is emitted from the chair to create a shroud of clean air about the head of any person sitting in the chair. The apparatus may be used to maintain a clean-air atmosphere about at least the head of an animal in a animal hospital or veterinary practice.

The apparatus 310 may have a throughput of from 20 to 80 Its or less. The apparatus may be able to maintain a shroud of clean air of between, say, 0.25 and 4 m³.

This embodiment is useful when attempting to maintain a ‘clean air’ environment. In a large space, for example a hospital ward, it is necessary to have a large throughput of air through the air cleaning apparatus. Such a large throughput requires commensurately large apparatus with a plurality of relatively intense UV lights to irradiate a large volume of gas. In many instances, large apparatus may be unwieldy or unwarranted, it may not fit into the available space or it may not be economically feasible.

If, for example, one patient in a ward requires clean air due to a suppressed immune system, an allergy or the like, whilst the other patients have no such requirements it is plainly wasteful to clean the air for the entire ward when it is only required by a single individual. The apparatus 101 of the current invention is therefore very useful. Other apparatus 1, 51, 101′ may be used in place of the apparatus 101.

A second use for the apparatus 1, 51, 101, 101′ is in the vacuum cleaning apparatus shown in FIG. 7. The vacuum cleaning apparatus comprises a housing A having front wheels B and a push handle C. The underside of the housing A has an open throat D leading to a fan unit E and thence to a collection bag F. The throat D contains a rotary brush G. A UV light unit H is present to irradiate air flowing through the throat D. The unit H may take any of the forms as shown in FIGS. 1 to 4B and are arranged to project ultraviolet light on to the brush G. In this embodiment, the grid 10 may be removed to allow UV light to irradiate the substrate S. If the grid 10 is not present, the device will comprise ‘tilt’ switches or other means which cut the power to the lights when the vacuum cleaner is turned upright to prevent accidental exposure of an individual to the UV radiation.

In use, the operator pushes the apparatus along the floor S and using controls not shown, activates the vacuum. The brush G rotates to clean the surface S. Air is drawn into the throat D and the particles collect in the collection unit F. Air passing through the unit H is treated by the ultraviolet radiation and then joins the main stream. Radiation is directed downwards to sterilise the surface of the substrate S, so killing any micro-organisms on the substrate. (Switches, not shown, may be present so that the unit H are energised once the apparatus is moving and deactivated when movement stops.)

In hospitals there is often a need for positive or negative pressure rooms. It is usual for such a room to be configured for either negative or positive pressure conditions but not alterable between the two.

The capital cost for building a positive or negative pressure room substantial. Utilising apparatus of the current invention can substantially reduce such costs.

In each case the room contains a bed for a patient and a bathroom. The room has a main entry/exit door system arranged to allow access with minimal effect on the atmosphere within the room. In each case, the corner of the room is a unit 200, as shown in FIG. 5.

The unit 200 provides a highly efficient device for controlling the risk of airborne infection in an atmosphere appropriate to the treatment of the patient.

FIGS. 8 and 9 show the typical layout of a room which can be configured for negative or positive pressure. It will be appreciated that by altering the direction of flow of the valve, the room can be readily changed from a positive to a negative pressure room. As is shown, in the negative pressure room excess air is exhausted to the outside of the room from the unit 200 and in the positive pressure room air is directed into the unit 200 from outside the room. In both cases, air is directed into the room via a centrally disposed conduit 1000.

FIG. 10 shows the use of air cleaning apparatus 1, 51, 101, 101′ in a unit U used in an operating theatre.

In use, the unit U is activated and air is passed therethrough to destroy airborne pathogens. The cleaned sterile air exits via the conduit C; to the hood H and then downwardly in laminar flow over the operating table T. In this way the working zone is kept sterile. The air is then collected in the floor level through A and recycled via the unit U.

A further use to which the air cleaning apparatus 1, 51, 101, 101′ may be put is in apparatus to isolate and destroy hazardous material. For example, hazardous material which is in or on a package or letter which has been sent through the post.

Referring to FIGS. 11, 12 and 13, the apparatus comprises a tall cabinet 501 of rectangular cross-sectional shape. In one sidewall there is a sealable entry door 502. A glass window is present 503. Various control devices 504 are present on the outside of the cabinet.

As shown most clearly in FIG. 12, the apparatus comprises an air cleaning and sensitising unit 510 in the bottom left half. To the right, there is a table 511 having a mesh 512 or similar surface adjacent the entry door 502. The unit 510 is of the type described above in relation to FIG. 5A. The table 511 may be viewed via the window 503. Two arms 513 linked to rubber gloves 514 are accessible from outside the cabinet and extend into the chamber to reach to the table 511. A shelf 515 is present to house plastic bags, scissors and similar instruments. A set of ultraviolet lamps 516 is present below the table 511, near the floor.

In use, pressurised air enters from the bottom of the cabinet and passes through the unit 510 to enter the chamber in the zone of the table 511. The air then passes over a package which has been placed on the table via the door 502. Using the gloves the operator manipulates the package and any external coating or powder is likely to be displaced. The powder will fall on to the ultra-violet light lamps 516 below the table 511, to destroy any pathogens located therein. Powder which does not settle on the lamps is carried by the pressurised air back to the cleaning and sensitising unit 510 and in so doing, passes over the ultra-violet lamps. In this way all the released material will be destroyed.

If the operator considers that the package may have further hazards, he could place it in one of the sealable bags on the shelf 515 and so isolate it from the chamber. It can then be removed via the door.

The chamber has an outlet duct in its ceiling which leads via another filter to the atmosphere and level of air removed is adjusted to ensure that there is always a negative pressure within the chamber.

The hazardous material will be any poison capable of being applied to or located within a package, including an envelope, and which is capable of being harmed, ideally killed by ultraviolet light.

The working surface is a table having perforations to allow the pressurised air to flow past. The table may have a flat surface or be of V section.

The invention is not limited to the embodiments shown. Baffles may be present to prevent UV radiation leakage such as a UV screen at the inlet and the outlet. A carbon filter may also be present at the outlet. Electronic controllers may be present to monitor the temperature of the lamps and intensity.

The apparatus 1, 51, 101, 101′ may be used to kill or at least render non-viable most, if not all, moulds, viruses and bacteria, including anthrax, TB, MRSA superbug, staphylococcus aureus, Escheria coli, streptococcus pneumonias. In tests it has been shown that it is possible to achieve at least a 4 or 5 log reduction in the amount of viable bacteria and/or viruses present using the apparatus 1, 51, 101, 101′ of the invention.

It will be appreciate that any of the variations of the apparatus 1, 51, 101, 101′ may be incorporated into a single unit. Apparatus 1, 51, 101, 101′ may be situated in series or parallel.

The upstream filter 9, 55, 105, 105′ may be of the photocatalyst-type. It may be coated (on a downstream, upstream or both sides) with TiO₂ or other substance which can cause generation of OH radicals when irradiated with UV light.

Sensors which detect the presence of gaseous or air-borne pollutants may be present.

The invention extends to all combinations and sub-combinations of individual features shown in the drawings and disclosed in the above description.

1-34. (canceled)

35. Apparatus for cleaning air, the apparatus comprising a chamber having an inlet and an outlet, through which air to be cleaned is passable, a plurality of UV light sources to irradiate the interior of the chamber and one or more UV transmissible shield members isolating the UV light sources, in use, from the air to be cleaned and wherein at least some of the UV light sources are mounted on a common mounting means such that said some of the UV light sources are removable together from the apparatus.

36. Apparatus according to claim 35, further comprising filter means upstream of said UV light sources.

37. Apparatus cleaning air, the apparatus comprising a chamber having an inlet and an outlet, through which air to be cleaned is passable, one or more UV light sources to irradiate the interior of the chamber and one or more UV transmissible shield members isolating the UV light sources, in use, from the air to be cleaned and filter means upstream of the or each UV light source, and wherein upstream of said filter means is located air flow equalising means which, in use, ensures that the flow of air through said filter means is substantially constant across the area of said filter means.

38. Apparatus according to claim 37, wherein at least the downstream surface of said air flow equalising means is at least partially UV reflective.

39. Apparatus for cleaning air, the apparatus comprising a chamber having an inlet and an outlet, through which air to be cleaned is passable, one or more UV light sources to irradiate the interior of the chamber and one or more UV transmissible shield members isolating the UV light sources, in use, from the air to be cleaned and filter means upstream of the or each UV light source, and wherein upstream of said filter means is located a UV reflector arranged to allow air to flow therethrough but to prevent UV light from passing therethrough at least a portion of incidental UV light being reflected towards said filter means.

40. Apparatus according to claim 39, wherein the UV reflector forms part of air flow equalising means which, in use, ensures that the flow of air through said filter means is substantially constant across the area of said filter means.

41. Apparatus according to claim 37, wherein said air flow equalising means comprises a plurality of angled air flow paths.

42. Apparatus according to claim 41, wherein the angle of the air flow paths is about 45° to the major flow axis of air passing through the apparatus.

43. Apparatus according to claim 37, wherein said air flow equalising means comprises a plural-layered structure each layer comprising a plurality of angled flow paths.

44. Apparatus according to claim 43, wherein the flow paths of adjacent layers of said air flow equalising means are not parallel to encourage, in use, air flowing therethrough to adopt a tortuous flow path.

45. Apparatus according to claim 40, wherein said air flow equalising means comprises a plural-layered structure each layer comprising a plurality of angled flow paths.

46. Apparatus according to claim 35, wherein the UV transmissible shield member or members is/are formed from quartz, fused silica, UV transmissible plastics or other suitable materials.

47. Apparatus according to claim 37, wherein the UV transmissible shield member or members is/are formed from quartz, fused silica, UV transmissible plastics or other suitable materials.

48. Apparatus according to claim 39, wherein the UV transmissible shield member or members is/are formed from quartz, fused silica, UV transmissible plastics or other suitable materials.

49. Apparatus according to claim 35, wherein the or each UV light source is a UV lamp, emitting light in the UV-C band.

50. Apparatus according to claim 35, wherein the or each UV light source is of non-circular cross-sectional shape.

51. Apparatus according to claim 37, wherein the or each UV light source is of non-circular cross-sectional shape.

52. Apparatus according to claim 39, wherein the or each UV light source is of non-circular cross-sectional shape.

53. Apparatus according to claim 35, wherein the UV light sources are arranged, in use, to provide a mean level of radiation in the chamber of above 10 mW cm²).

54. Apparatus according to claim 53, wherein the UV light sources are arranged, in use, to provide a mean level of radiation in the chamber of at or above 60 mWcm⁻².

55. Apparatus according to claim 35, wherein the UV transmissible shield member provides at least part of a boundary defining the flow path of the air to be cleaned.

56. Apparatus according to claim 55, wherein the UV transmissible shield member is a tube mounted within the chamber, the air to be cleaned being flowable thorough the tubular shield member, the UV light sources being located between the chamber wall and the shield member.

57. Apparatus according to claim 55, wherein the UV transmissible shield member provides at least one wall of the chamber, the or each UV light source being located outside of the to-be-cleaned air flow path.

58. Apparatus according to claim 57, wherein the or each UV light source is located adjacent a UV reflective surface to reflect impinging UV radiation into the chamber.

59. Apparatus according to claim 35, wherein the UV transmissible shield member(s) comprises one or more tubes which extend across the chamber, a UV light source being mounted within the or each tubular UV transmissible shield members.

60. Apparatus according to claim 35, wherein the chamber comprises one or more walls which comprise a UV reflective surface.

61. Apparatus according to claim 60, wherein said one or more walls comprising a UV reflective surface may be shaped to present a concave surface as viewed from the major axis of the flowing air.

62. Apparatus according to claim 35, further comprising filter means situated across the outlet.

63. Apparatus according to claim 62, wherein said outlet filter means comprises a HEPA filter element.

64. Apparatus according to claim 62, wherein said outlet filter means comprises a combustible frame, such as one fabricated from wood, a derivative of wood or the like.

65. Apparatus according to claim 37, wherein said upstream, inlet filter means is fabricated from a UV transmissible material.

66. Apparatus according to claim 37, wherein said upstream, inlet filter means is coated and/or impregnated with an antimicrobial or biostatic substance.

67. Apparatus according to claim 35, further comprising a component made of a material which releases hydroxyl radials.

68. Apparatus according to claim 37, further comprising a component made of a material which releases hydroxyl radials.

69. Apparatus according to claim 39, further comprising a component made of a material which releases hydroxyl radials.

70. Apparatus according to claim 35, comprising a plurality of UV light sources, one power supply being present for each pair of UV light sources.

71. Apparatus according to claim 37, comprising a plurality of UV light sources, one power supply being present for each pair of UV light sources.

72. Apparatus according to claim 39, comprising a plurality of UV light sources, one power supply being present for each pair of UV light sources.

73. Apparatus according to claim 70, wherein the or each power supply is tunable to deliver less or more power to the UV light sources to which it is connected.

74. Apparatus according to claim 70, further comprising UV monitoring means arranged to control the or each power supply to increase or decrease the power supplied, thereby ensuring that the UV irradiation within the chamber is monitored and/or kept within tolerable limits.

75. Apparatus for cleaning air, the apparatus) comprising a chamber, having an inlet and an outlet, through which air to be cleaned is passable, a plurality of UV light sources to irradiate the interior of the chamber and one or more UV transmissible shield members isolating the UV light sources, in use, from the air to be cleaned and wherein two or more UV light sources are supplied with power from a tunable power source, said two or more UV light sources having UV monitoring means associated therewith, said UV monitoring means being arranged in operative feedback control of the power source to ensure UV output from the UV light sources is within tolerable limits.

76. A portable air cleaning unit, the unit comprising a housing in which is located air cleaning apparatus according to claim 35 and air movement means operable to cause air to flow from the inlet to the outlet of the air cleaning apparatus.

77. A portable air cleaning unit, the unit comprising a housing in which is located air cleaning apparatus according to claim 37 and air movement means operable to cause air to flow from the inlet to the outlet of the air cleaning apparatus.

78. A method of cleaning air, the method comprising moving air through air cleaning apparatus as claimed in claim 35 and expelling cleaned air.
